(19) United States
(12) Patent Application Publication
(10) Pub. No.: US 2021/0338720 A1
(43) Pub. Date: Nov. 4, 2021

(54) COMPOSITIONS AND METHODS FOR THE TREATMENT OF MASTITIS

(71) Applicants: Tinu Mary Samuel, La Tour De Peilz (CH); lrma Silva Zolezzi, Singapore (SG)

(72) Inventors: Tinu Mary Samuel, La Tour De Peilz (CH); lrma Silva Zolezzi, Singapore (SG)

(21) Appl. No.: 17/270,539

(22) PCT Filed: Aug. 27, 2019
(86) PCT No.: PCT/EP2019/072794

§ 371 Date: Feb. 23, 2021

(30) Foreign Application Priority Data
Aug. 27, 2018 (EP) 18191022.5

(51) Int. Cl. A61K 33/32; A61K 33/26; A61K 33/06; A61K 33/34; A61K 33/30; A61K 33/04; A61K 33/42; A61K 31/355; A61K 31/685; A61K 38/40; A61K 38/38; A61K 31/202; A61P 15/14

(52) U.S. Cl. CPC A61K 33/32; A61K 33/26; A61K 33/06; A61K 33/34; A61K 33/30; A61K 33/04; A61P 15/14; A61K 31/355; A61K 31/685; A61K 38/40; A61K 38/38; A61K 31/202; A61K 33/42

(57) ABSTRACT

A mineral selected from the group consisting of iron, manganese and magnesium, or combination of two of more thereof; An n-3 fatty acid selected from the group consisting of docosahexaenoic acid (DHA) and 18:3 n-3 octadecatrienoic acid (alpha-linolenic acid); or a protein selected from the group consisting of alpha-lactalbumin, lactoferrin and albumin for use in treating or preventing mastitis in a subject.

### FIELD OF THE INVENTION

The present invention relates to compositions for use in treating or preventing mastitis, for example sub-clinical mastitis, in a subject. In particular, the invention relates to the use of minerals such as iron, manganese and magnesium; fatty acids such as docosahexaenoic acid (DHA) and 18:3 n-3 octadecatrienoic acid (alpha-linolenic acid); and proteins such as alpha-lactalbumin, lactoferrin and albumin in treating or preventing mastitis, in particular sub-clinical mastitis.

### BACKGROUND TO THE INVENTION

WHO recommends that infants should be exclusively breastfed for the first six months of life to achieve optimal growth, development and health and continued breast feeding until 2 years of age. According to WHO, exclusive breastfeeding means that the infant receives only breast milk (no other liquids or solids are given—not even water—with the exception of oral rehydration solution, or drops/syrups of vitamins, minerals or medicines). WHO also recommends early initiation of breastfeeding as this may is critical to newborn survival and to establishing breastfeeding over the long term.

Mastitis is an inflammation of the mammary gland tissue, which can be classified as sub-clinical or clinical depending on the degree of inflammation.

Mastitis may occur at any time during lactation and is experienced by up to about 33% of lactating women. Occurrence is particularly prevalent during the second and third week post-partum.

Sub-clinical mastitis (SCM) is an inflammatory condition of the lactating breast that is understood to be caused by milk stasis and/or infection, and has been associated with elevated risk of lactation failure and poor infant weight gain.

Staphylococcus infections, in particular S. aureus and S. epidermidis infections, are understood to be a primary cause of mastitis.

Mastitis can result in curtailment or even lack of initiation of breast-feeding of an infant.

Furthermore, the composition of breast milk may change during mastitis, for example increasing in content of sodium and inflammatory mediators, which may adversely affect the nutrition provided to the infant.

Current treatment of mastitis typically involves the administration of antibiotics. However, wide-spread use of antibiotics presents several challenges, including ineffectiveness due to antibiotic resistance, the creation of multiple-antibiotic resistant strains of bacteria, the formation of biofilms, vaginal candidiasis and antibiotic-associated diarrhoea.

Moreover, it has been indicated that there is insufficient evidence to support the effectiveness of antibiotic therapy for the treatment of lactational mastitis (Jahanfar, S. et al. (2013) Cochrane Database Syst Rev 28: CD005458).

Accordingly, there is a significant need for improved methods of treating and preventing mastitis.

### SUMMARY OF THE INVENTION

The inventors have surprisingly found that a number of minerals in the milk of women with sub-clinical mastitis, such as iron, manganese and magnesium, are present at abnormal concentrations.

Concentrations of sodium and potassium in milk are commonly used in the diagnosis of sub-clinical mastitis. For example, a number of studies have found that Na:K ratios in the milk of healthy women at 1 month post-partum generally average 0.6 or less. This corresponds to average human milk sodium and potassium concentrations ranging between 5-6 mmol/L and 13-14 mmol/L, respectively. In contrast, the mean sodium concentration in mastitis milk is greater than 16 mmol/L. Accordingly, a Na:K ratio of less than or equal to 0.6 is considered to be normal; a Na:K ratio of greater than 0.6 but less than or equal to 1.0 is considered to be moderately raised; and a Na:K ratio of greater than 1.0 is considered to be greatly raised.

Another study suggests that a normal drop in [Na+] is highly predictive of successful lactation, although a prolonged elevation of [Na+] signifies impaired lactogenesis with a high risk of failure.

The inventors have studied the concentrations of additional components in the milk of women with Na:K ratios greater than 0.6 and compared these to the concentrations found in the milk of normal women. Compositional differences have been identified in a number of minerals.

Specifically, the inventors have found that women with sub-clinical mastitis have: higher concentrations of minerals such as iron, manganese, magnesium, copper, zinc and selenium; and lower concentrations of minerals such as calcium and phosphorous in their milk in comparison to normal women.

The minerals that exhibit lower concentrations in the milk of women with sub-clinical mastitis (e.g. calcium and phosphorous) correlate with deficiencies that may be causing or contributing to the sub-clinical mastitis. Supplementation with such minerals may therefore prevent or treat the sub-clinical mastitis. In addition, the minerals with higher concentrations in the milk of women with sub-clinical mastitis (e.g. iron, manganese, magnesium, copper, zinc and selenium) correlate with the natural use of such minerals in countering infection and/or inflammation. Supplementation with such minerals may therefore be beneficial to the natural fight against infection and inflammation, thereby preventing or treating the sub-clinical mastitis.

Without wishing to be bound by theory, this rationale is supported by the knowledge that selenium improves antibacterial activity in milk and that selenium supplementation improves symptoms associated with mastitis in cows; similarly, copper and zinc have also been shown to reduce mastitis symptoms in cows and to enhance the immune system (O'Rourke, D. (2009) Irish Veterinary Journal 62 Supplement: 15-20).

The inventors believe that the elevated mineral concentrations observed in their data may result from increased uptake or hyper-accumulation from serum as part of host defense mechanisms to combat inflammation, which is consistent with roles for, for example, iron manganese and magnesium in immune function and countering inflammation (Rahmani, S. et al. (2015) J Nutr Food Sci 5: 1; Son, E. W. et al. (2007) Arch Pharm Res 30: 743-749; Maggini, S. et al. (2007) Br J Nutr 98 Suppl 1: S29-35; Tam, M. et al. (2003) Eur J Clin Nutr 57: 1193-1197; Kim, D. J. et al. (2010) Diabetes Care 33: 2604-2610; King, D. E. et al. (2005) J Am Coll Nutr 24: 166-171; Song, Y. et al. (2007) Am J Clin Nutr 85: 1068-1074).

The role of certain dietary minerals in prevention of subclinical mastitis (for example iron, manganese, magnesium, copper, calcium and phosphorus) has been also confirmed by the inventors in the experiments described in Example 2.

The inflammatory state associated with sub-clinical mastitis alters the levels and ratios of fatty acids in milk. In particular, the inventors have discovered that fatty acid concentrations vary in the milk of women with sub-clinical mastitis. For example, the inventors found that the n-3 fatty acids docosahexaenoic acid (DHA) and 18:3 n-3 octadecatrienoic acid (alpha-linolenic acid) are present at lower concentrations in the milk of women with sub-clinical mastitis in comparison to normal women. The inventors also found higher n-6:n-3 ratios and higher arachidonic acid (ARA):DHA ratios in the milk of women with sub-clinical mastitis in comparison to normal women.

The higher n-6:n 3 ratio, ARA:DHA ratio and lower amounts of DHA all point towards a pro-inflammatory state. Supplementation with n-3 fatty acids, such as DHA and alpha-linolenic acid, may therefore also be used in treating or preventing the sub-clinical mastitis in a similar manner to that disclosed herein with respect to minerals such as calcium and phosphorous.

In addition, the inventors have discovered that alpha-lactalbumin, lactoferrin and albumin are present at higher concentrations in the milk of women with sub-clinical mastitis in comparison to normal women. Supplementation with these proteins may therefore also be used in treating or preventing the sub-clinical mastitis in a similar manner to that disclosed herein with respect to minerals such as iron, manganese, magnesium, copper, zinc and selenium.

Accordingly, in one aspect the invention provides a mineral selected from the group consisting of iron, manganese, magnesium, and a combination of two of more thereof, for use in treating or preventing mastitis in a subject.

In another aspect, the invention provides iron for use in treating or preventing mastitis in a subject, preferably wherein the iron is in a combination with manganese and/or magnesium.

In another aspect, the invention provides iron for use in treating or preventing mastitis in a subject, wherein the iron is administered to the subject with manganese and/or magnesium. In one embodiment, the iron is administered to the subject simultaneously, sequentially or separately with manganese and/or magnesium, preferably simultaneously.

In another aspect, the invention provides manganese for use in treating or preventing mastitis in a subject, preferably wherein the manganese is in combination with iron and/or magnesium.

In another aspect, the invention provides manganese for use in treating or preventing mastitis in a subject, wherein the manganese is administered to the subject with iron and/or magnesium. In one embodiment, the manganese is administered to the subject simultaneously, sequentially or separately with iron and/or magnesium, preferably simultaneously.

In another aspect, the invention provides magnesium for use in treating or preventing mastitis in a subject, preferably wherein the magnesium is in combination with iron and/or manganese.

In another aspect, the invention provides magnesium for use in treating or preventing mastitis in a subject, wherein the magnesium is administered to the subject with iron and/or manganese. In one embodiment, the magnesium is administered to the subject simultaneously, sequentially or separately with iron and/or manganese, preferably simultaneously.

In another aspect, the invention provides a combination of two or more minerals selected from the group consisting of (a) iron; (b) manganese; and (c) magnesium for use in treating or preventing mastitis in a subject.

In one embodiment, two or more of (a), (b) and (c) are administered to the subject simultaneously, sequentially or separately.

In a preferred embodiment, two or more of (a), (b) and (c) are administered to the subject simultaneously.

In another aspect, the invention provides a composition comprising one or more minerals selected from the group consisting of iron, manganese and magnesium for use in treating or preventing mastitis in a subject.

In another aspect, the invention provides a method for treating or preventing mastitis, wherein the method comprises administering one or more minerals selected from the group consisting of iron, manganese and magnesium to a subject in need thereof.

In one embodiment, two or more of iron, manganese and magnesium are administered to the subject simultaneously, sequentially or separately.

In a preferred embodiment, two or more of iron, manganese and magnesium are administered to the subject simultaneously.

In one embodiment, the combination or composition comprises iron and manganese. In one embodiment, the subject is administered iron and manganese, preferably simultaneously, sequentially or separately, more preferably simultaneously.

In one embodiment, the combination or composition comprises iron, manganese and magnesium. In one embodiment, the subject is administered iron, manganese and magnesium, preferably simultaneously, sequentially or separately, more preferably simultaneously.

In one embodiment, the mineral is in combination with one or more further minerals selected from the group consisting of copper, zinc, selenium, calcium and phosphorus. In one embodiment, the combination or composition further comprises one or more minerals selected from the group consisting of copper, zinc, selenium, calcium and phosphorus. In one embodiment, the iron, manganese, magnesium, or combination of two or more thereof is administered to the subject with one or more minerals selected from the group consisting of copper, zinc, selenium, calcium and phosphorus, preferably simultaneously, sequentially or separately, more preferably simultaneously.

In one embodiment, the mineral is in combination with vitamin E. In one embodiment, the combination or composition further comprises vitamin E. In one embodiment, the iron, manganese, magnesium, or combination of two or more thereof is administered to the subject with vitamin E, preferably simultaneously, sequentially or separately, more preferably simultaneously.

In one embodiment, the combination or composition comprises iron, manganese, and one or more minerals selected from the group consisting of copper, zinc, selenium, calcium and phosphorus. In one embodiment, the subject is administered iron, manganese, and one or more minerals selected from the group consisting of copper, zinc, selenium, calcium and phosphorus, preferably simultaneously, sequentially or separately, more preferably simultaneously.

In one embodiment, the combination or composition comprises iron, manganese and vitamin E. In one embodiment, the subject is administered iron, manganese and vitamin E, preferably simultaneously, sequentially or separately, more preferably simultaneously.

In one embodiment, the combination or composition comprises iron, manganese, magnesium, and one or more minerals selected from the group consisting of copper, zinc, selenium, calcium and phosphorus. In one embodiment, the subject is administered iron, manganese, magnesium, and one or more minerals selected from the group consisting of copper, zinc, selenium, calcium and phosphorus, preferably simultaneously, sequentially or separately, more preferably simultaneously.

In one embodiment, the combination or composition comprises iron, manganese, magnesium and vitamin E. In one embodiment, the subject is administered iron, manganese, magnesium and vitamin E, preferably simultaneously, sequentially or separately, more preferably simultaneously.

In a preferred embodiment, the combination or composition comprises iron, manganese, copper, zinc, selenium and vitamin E. In a preferred embodiment, the subject is administered iron, manganese, copper, zinc, selenium and vitamin E, preferably simultaneously, sequentially or separately, more preferably simultaneously.

In one embodiment, the mineral is in combination with an n-3 fatty acid, preferably a fatty acid selected from the group consisting of docosahexaenoic acid (DHA) and 18:3 n-3 octadecatrienoic acid (alpha-linolenic acid). In one embodiment, the combination or composition further comprises an n-3 fatty acid, preferably a fatty acid selected from the group consisting of docosahexaenoic acid (DHA) and 18:3 n-3 octadecatrienoic acid (alpha-linolenic acid). In one embodiment, the iron, manganese, magnesium, or combination of two or more thereof is administered to the subject with an n-3 fatty acid (preferably a fatty acid selected from the group consisting of docosahexaenoic acid (DHA) and 18:3 n-3 octadecatrienoic acid (alpha-linolenic acid)), preferably simultaneously, sequentially or separately, more preferably simultaneously.

In one embodiment, the combination or composition comprises iron, manganese and an n-3 fatty acid, preferably a fatty acid selected from the group consisting of docosahexaenoic acid (DHA) and 18:3 n-3 octadecatrienoic acid (alpha-linolenic acid). In one embodiment, the subject is administered iron, manganese and an n-3 fatty acid (preferably a fatty acid selected from the group consisting of docosahexaenoic acid (DHA) and 18:3 n-3 octadecatrienoic acid (alpha-linolenic acid)), preferably simultaneously, sequentially or separately, more preferably simultaneously.

In one embodiment, the combination or composition comprises iron, manganese, magnesium and an n-3 fatty acid, preferably a fatty acid selected from the group consisting of docosahexaenoic acid (DHA) and 18:3 n-3 octadecatrienoic acid (alpha-linolenic acid). In one embodiment, the subject is administered iron, manganese, magnesium and an n-3 fatty acid (preferably a fatty acid selected from the group consisting of docosahexaenoic acid (DHA) and 18:3 n-3 octadecatrienoic acid (alpha-linolenic acid)), preferably simultaneously, sequentially or separately, more preferably simultaneously.

In one embodiment, the mineral is in combination with a protein selected from the group consisting of alpha-lactalbumin, lactoferrin and albumin. In one embodiment, the combination or composition further comprises a protein selected from the group consisting of alpha-lactalbumin, lactoferrin and albumin. In one embodiment, the iron, manganese, magnesium, or combination of two or more thereof is administered to the subject with a protein selected from the group consisting of alpha-lactalbumin, lactoferrin and albumin, preferably simultaneously, sequentially or separately, more preferably simultaneously.

In one embodiment, the combination or composition comprises iron, manganese and a protein selected from the group consisting of alpha-lactalbumin, lactoferrin and albumin. In one embodiment, the subject is administered iron, manganese and a protein selected from the group consisting of alpha-lactalbumin, lactoferrin and albumin, preferably simultaneously, sequentially or separately, more preferably simultaneously.

In one embodiment, the combination or composition comprises iron, manganese, magnesium and a protein selected from the group consisting of alpha-lactalbumin, lactoferrin and albumin. In one embodiment, the subject is administered iron, manganese, magnesium and a protein selected from the group consisting of alpha-lactalbumin, lactoferrin and albumin, preferably simultaneously, sequentially or separately, more preferably simultaneously.

In one embodiment, the mineral is in combination with phosphatidylcholine and/or lecithin. In one embodiment, the combination or composition further comprises phosphatidylcholine and/or lecithin. In one embodiment, the iron, manganese, magnesium, or combination of two or more thereof is administered to the subject with phosphatidylcholine and/or lecithin, preferably simultaneously, sequentially or separately, more preferably simultaneously.

In one embodiment, the combination or composition comprises iron and manganese; and phosphatidylcholine and/or lecithin. In one embodiment, the subject is administered iron and manganese; and phosphatidylcholine and/or lecithin, preferably simultaneously, sequentially or separately, more preferably simultaneously.

In one embodiment, the combination or composition comprises iron and manganese and magnesium; and phosphatidylcholine and/or lecithin. In one embodiment, the subject is administered iron and manganese and magnesium; and phosphatidylcholine and/or lecithin, preferably simultaneously, sequentially or separately, more preferably simultaneously.

In another aspect, the invention provides an n-3 fatty acid for use in treating or preventing mastitis in a subject.

In one embodiment, the fatty acid is selected from the group consisting of docosahexaenoic acid (DHA) and 18:3 n-3 octadecatrienoic acid (alpha-linolenic acid).

In another aspect, the invention provides a protein selected from the group consisting of alpha-lactalbumin, lactoferrin and albumin for use in treating or preventing mastitis in a subject.

In one embodiment, the iron, manganese, magnesium or combination, fatty acid or protein is in the form of a composition.

In one embodiment, the composition is a nutritional composition or a pharmaceutical composition, preferably a nutritional composition.

In one embodiment, the composition is a maternal nutritional composition, preferably for use during lactation and/or pregnancy.

In one embodiment, the iron, manganese, magnesium, combination, fatty acid, protein or composition is in the form of a tablet, gel capsule, powder, maternal milk powder, food product, liquid format (e.g. ready to drink format) and/or beverage.

In one embodiment, the mastitis is sub-clinical mastitis or clinical mastitis.

In a preferred embodiment, the mastitis is sub-clinical mastitis.

In one embodiment, the subject is at risk of suffering from sub-clinical mastitis or clinical mastitis.

In one embodiment, the risk of suffering from mastitis (such as sub-clinical mastitis or clinical mastitis) is indicated by the presence of one or more risk factors selected from the group consisting of family history of sub-clinical mastitis or clinical mastitis, breast-feeding attachment difficulties, mother-infant separation (e.g. separation of greater than 24 h), blocked duct, milk stasis, cracked nipples, pre-lacteal feeds, milk oversupply, breast engorgement, feeding from alternate breasts on consecutive feeds, infant mouth abnormalities, a short infant frenulum, maternal use of antibiotics, previous history of mastitis in the subject, maternal stress, delivery in private versus public hospital and the presence of Staphylococcus aureus in milk.

In one embodiment, the subject is a human e.g. a woman who is desiring to get pregnant, who is pregnant or who is lactating.

In one embodiment, the subject is a livestock animal or a companion animal. In one embodiment, the subject is a cow or dog. In another embodiment, the subject is a rat or mouse.

In one embodiment, the treatment or prevention increases the probability of initiating and/or continuing breastfeeding by the subject.

In one embodiment, the treatment or prevention increases the probability of the subject exclusively breast-feeding her infant.

In one embodiment, the treatment or prevention increases the duration (length of time e.g. number of days, weeks, months) of breastfeeding by the subject.

In one embodiment, the subject is able to breast-feed for at least 4 months, preferably 4-24 months, optionally 4-6 months.

In one embodiment, the subject is able to breast-feed for at least 6 months, preferably 6-24 months.

In one embodiment, the treatment or prevention increases the quality of the subject's breast milk.

In one embodiment, the treatment or preventing increases the quantity of the subject's breast milk.

In another aspect, the invention provides a composition for use in treating or preventing mastitis in a subject, wherein the composition comprises a mineral, fatty acid, protein or combination as defined herein.

In another aspect, the invention provides a combination of (a) iron; (b) manganese; (c) copper; (d) zinc; (e) selenium; and (f) vitamin E for use in treating or preventing mastitis in a subject, preferably wherein (a)-(f) are administered to the subject simultaneously, sequentially or separately, more preferably wherein (a)-(f) are administered to the subject simultaneously.

In another aspect, the invention provides a composition comprising iron, manganese, copper, zinc, selenium and vitamin E for use in treating or preventing mastitis in a subject.

In another aspect, the invention provides a method for treating or preventing mastitis, wherein the method comprises administering iron, manganese, copper, zinc, selenium and vitamin E to a subject in need thereof, preferably wherein the iron, manganese, copper, zinc, selenium and vitamin E are administered to the subject simultaneously, sequentially or separately, more preferably wherein the iron, manganese, copper, zinc, selenium and vitamin E are administered to the subject simultaneously.

In another aspect, the invention provides a mineral selected from the group consisting of iron, manganese, magnesium, and a combination of two of more thereof, for use in reducing the risk of mastitis in a subject.

In another aspect, the invention provides a combination of two or more minerals selected from the group consisting of (a) iron; (b) manganese; and (c) magnesium for use in reducing the risk of mastitis in a subject.

In another aspect, the invention provides iron for use in reducing the risk of mastitis in a subject, preferably wherein the iron is administered to the subject simultaneously, sequentially or separately with manganese and/or magnesium.

In another aspect, the invention provides manganese for use in reducing the risk of mastitis in a subject, preferably wherein the manganese is administered to the subject simultaneously, sequentially or separately with iron and/or magnesium.

In another aspect, the invention provides magnesium for use in reducing the risk of mastitis in a subject, preferably wherein the magnesium is administered to the subject simultaneously, sequentially or separately with iron and/or manganese.

In another aspect, the invention provides a composition comprising one or more minerals selected from the group consisting of iron, manganese and magnesium for use in reducing the risk of mastitis in a subject.

In another aspect, the invention provides a method for reducing the risk of mastitis, wherein the method comprises administering one or more minerals selected from the group consisting of iron, manganese and magnesium to a subject in need thereof.

In another aspect, the invention provides a combination of (a) iron; (b) manganese; (c) copper; (d) zinc; (e) selenium; and (f) vitamin E for use in reducing the risk of mastitis in a subject.

In another aspect, the invention provides a composition comprising iron, manganese, copper, zinc, selenium and vitamin E for use in reducing the risk of mastitis in a subject.

In another aspect, the invention provides an n-3 and/or n-6 fatty acid for use in reducing the risk of mastitis in a subject.

In another aspect, the invention provides a protein selected from the group consisting of alpha-lactalbumin, lactoferrin and albumin for use in reducing the risk of mastitis in a subject.

In another aspect, the invention provides a method for reducing the risk of mastitis, wherein the method comprises administering iron, manganese, copper, zinc, selenium and vitamin E to a subject in need thereof, preferably wherein the iron, manganese, copper, zinc, selenium and vitamin E are administered to the subject simultaneously, sequentially or separately, more preferably wherein the iron, manganese, copper, zinc, selenium and vitamin E are administered to the subject simultaneously.

### DESCRIPTION OF THE DRAWINGS

FIG. 1

Comparison of iron concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 2

Comparison of manganese concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 3

Comparison of magnesium concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 4

Comparison of copper concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 5

Comparison of zinc concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 6

Comparison of selenium concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 7

Comparison of calcium concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 8

Comparison of phosphorous concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 9

Comparison of docosahexaenoic acid (DHA) concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 10

Comparison of 18:3 n-3 octadecatrienoic acid (alpha-linolenic acid) concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 11

Comparison of alpha-lactalbumin concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 12

Comparison of lactoferrin concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

FIG. 13

Comparison of albumin concentrations between the milk of mothers with sub-clinical mastitis and the milk of normal mothers at 6 time-points post-partum (V1=0-3 d; V2=17±3 d; V3=30±3 d; V4=60±5 d; V5=90±5 d; and V6=120±5 d).

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Mastitis

Mastitis is an inflammation of the mammary gland tissue, which can be classified as sub-clinical or clinical depending on the degree of inflammation.

Clinical mastitis is a form of mastitis associated with reduced milk secretion, visible signs of inflammation of the breast and, changes in the appearance of milk, which may be accompanied by systemic signs. Sub-clinical mastitis is a form of mastitis characterised by reduced milk secretion and a high milk bacterial count in the absence of evident inflammatory changes, including pain (Fernandez, L. et al. (2014) Beneficial Microbes 5: 169-183).

Concentrations of sodium and potassium in milk are commonly used in the diagnosis of sub-clinical mastitis. For example, a number of studies have found that Na:K ratios in the milk of healthy women at 1 month post-partum generally average 0.6 or less. This corresponds to average human milk sodium and potassium concentrations ranging between 5-6 mmol/L and 13-14 mmol/L, respectively. In contrast, the mean sodium concentration in mastitis milk is greater than 16 mmol/L. Accordingly, a Na:K ratio of less than or equal to 0.6 is considered to be normal; a Na:K ratio of greater than 0.6 but less than or equal to 1.0 is considered to be moderately raised; and a Na:K ratio of greater than 1.0 is considered to be greatly raised.

Mastitis may occur at any time during lactation and is experienced by up to about 33% of lactating women. Occurrence is particularly prevalent during the second and third week post-partum.

Sub-clinical mastitis (SCM) is an inflammatory condition of the lactating breast that is understood to be caused by milk stasis and/or infection, and has been associated with elevated risk of lactation failure and poor infant weight gain.

Staphylococcus infections, in particular S. aureus and S. epidermidis infections, are understood to be a primary cause of mastitis.

Mastitis can result in curtailment or even lack of initiation of breast-feeding of an infant. Furthermore, the composition of breast milk may change during mastitis, for example increasing in content of sodium and inflammatory mediators, which may adversely affect the nutrition provided to the infant.

Mineral

In one aspect, the invention provides a mineral selected from the group consisting of iron, manganese, magnesium, and a combination of two of more thereof, for use in treating or preventing mastitis in a subject.

In one embodiment, the mineral is in combination with one or more further minerals selected from the group consisting of copper, zinc, selenium, calcium and phosphorus.

The minerals may be used in any form suitable for ingestion by animals, preferably humans (e.g. are non-toxic). The minerals may be used, for example in compositions such as nutritional compositions, in any appropriate amount. The skilled person will be able to determine appropriate amounts depending on the desired dosage of the mineral. Dosages may depend on factors such as the age, size and health status of the woman to whom they are administered, on her lifestyle, as well as on her genetic heritage. Dosages may be in line with the recommended daily intakes (RDA) developed by organisations such as the Food and Nutrition Board of the National Academy of Sciences.

The skilled person can readily determine an appropriate dose of one of the agents of the invention to administer to a subject without undue experimentation. Typically, a physician will determine the actual dosage that will be most suitable for an individual subject and it will depend on a variety of factors including the activity of the specific agent employed, the metabolic stability and length of action of that agent, the age, body weight, general health, sex, diet, mode and time of administration, rate of excretion, drug combination, the severity of the particular condition and the individual undergoing therapy. There can of course be individual instances where higher or lower dosage ranges are merited.

In one embodiment, the dosage of iron is about 2.7-45, 5-25 or 9-10 mg/day. A dosage of about 9-10 mg/day may be preferred for breast-feeding women.

In another embodiment, the dosage of iron is about 30-60 mg/day. A dosage of about 30-60 mg/day may be preferred for pregnant women.

In one embodiment, the dosage of iron is at least 9.1 mg/day. In a further embodiment, the dosage of iron is at least 9.5 mg/day. In a still further embodiment, the dosage of iron is ranging from 9.5 to 60 mg/day, for example from 9.5 to 50 mg/day, for example 9.5 to 40 mg/day.

In one embodiment, the dosage of iron for a lactating woman is at least 9.1 mg/day. In a further embodiment, the dosage of iron is at least 9.5 mg/day. In a still further embodiment, the dosage of iron is ranging from 9.5 to 60 mg/day, for example from 9.5 to 30 mg/day, for example 9.5 to 20 mg/day.

In one embodiment, the dosage of iron is at least 11.6 mg/day. In a further embodiment, the dosage of iron is at least 12 mg/day. In a still further embodiment, the dosage of iron is ranging from 12 to 60 mg/day, for example from 12 to 50 mg/day, for example 12 to 40 mg/day.

In one embodiment, the dosage of iron for a lactating woman is at least 11.6 mg/day. In a further embodiment, the dosage of iron is at least 12 mg/day. In a still further embodiment, the dosage of iron is ranging from 12 to 60 mg/day, for example from 12 to 30 mg/day, for example 12 to 20 mg/day.

The iron may be comprised in any form suitable for ingestion by a woman such as a pregnant woman, a woman trying to conceive or a lactating woman. For example, iron may be comprised in the form of iron sulfate, iron citrate, iron choline citrate, iron ammonium citrate, iron chloride, iron fumarate, iron gluconate, iron pyroposphate or a mixture thereof.

In one embodiment, the dosage of manganese is about 1.8-11, 2-3 or 2.5-2.7 mg/day. A dosage of about 2.5-2.7 mg/day may be preferred for breast-feeding women. A dosage of about 1.9-2.1 mg/day may be preferred for pregnant women.

In one embodiment, the dosage of manganese is at least 2.1 mg/day. In a further embodiment, the dosage of manganese is at least 2.3 mg/day. In a still further embodiment, the dosage of managese is ranging from 2.1 to 4 mg/day, for example from 2.3 to 3.5 mg/day.

In one embodiment, the dosage of manganese for a lactating woman is at least 2.1 mg/day. In a further embodiment, the dosage of manganese is at least 2.3 mg/day. In a still further embodiment, the dosage of managese is ranging from 2.1 to 4 mg/day, for example from 2.3 to 3.5 mg/day.

In one embodiment, the dosage of manganese is at least 2.6 mg/day. In a further embodiment, the dosage of manganese is at least 3.0 mg/day. In a still further embodiment, the dosage of managese is ranging from 2.6 to 4 mg/day, for example from 3.0 to 3.5 mg/day.

In one embodiment, the dosage of manganese for a lactating woman is at least 2.6 mg/day. In a further embodiment, the dosage of manganese is at least 3.0 mg/day. In a still further embodiment, the dosage of managese is ranging from 2.6 to 4 mg/day, for example from 3.0 to 3.5 mg/day.

The manganese may be comprised in any form suitable for ingestion by a woman such as a pregnant woman, a woman trying to conceive or a lactating woman. For example, manganese may be comprised in the form of manganese gluconate, manganese sulfate, manganese ascorbate, manganese amino acid chelates, manganese aspartate, manganese picolinate, manganese fumarate, manganese malate, manganese succinate, manganese citrate or a mixture thereof.

In one embodiment, the dosage of magnesium is about 35-350, 200-350 or 300-350 mg/day. A dosage of about 300-350 mg/day may be preferred for breast-feeding women.

In one embodiment, the dosage of magnesium is at least 270 mg/day. In a further embodiment, the dosage of magnesium is at least 300 mg/day. In a still further embodiment, the dosage of magnesium is ranging from 270 to 350 mg/day, for example from 300 to 350 mg/day.

In one embodiment, the dosage of magnesium for a lactating woman is at least 270 mg/day. In a further embodiment, the dosage of magnesium is at least 300 mg/day. In a still further embodiment, the dosage of magnesium is ranging from 270 to 350 mg/day, for example from 300 to 350 mg/day.

In one embodiment, the dosage of magnesium is at least 302 mg/day. In a further embodiment, the dosage of magnesium is at least 305 mg/day. In a still further embodiment, the dosage of magnesium is ranging from 302 to 350 mg/day, for example from 305 to 350 mg/day.

In one embodiment, the dosage of magnesium for a lactating woman is at least 302 mg/day. In a further embodiment, the dosage of magnesium is at least 305 mg/day. In a still further embodiment, the dosage of magnesium is ranging from 302 to 350 mg/day, for example from 305 to 350 mg/day.

The magnesium may be comprised in any form suitable for ingestion by a woman such as a pregnant woman, a woman trying to conceive or a lactating woman. For example, magnesium may be comprised in the form of magnesium chloride, magnesium citrate, magnesium sulfate, magnesium oxide, magnesium hydroxide, magnesium amino acid chelates (e.g. chelates of glycinate, lysinate, orotate, taurate, aspartate, threonate and/or malate) or a mixture thereof.

In one embodiment, the dosage of copper is about 0.1-10, 0.1-2 or 0.5-1.5 mg/day.

In one embodiment, the dosage of copper is at least 1.250 mg/day. In a further embodiment, the dosage of copper is at least 1.30 mg/day. In a still further embodiment, the dosage of copper is ranging from 1.250 to 10 mg/day, for example from 1.30 to 2 mg/day, for example from 1.30 to 1.50 mg/day.

In one embodiment, the dosage of copper for a lactating woman is at least 1.250 mg/day. In a further embodiment, the dosage of copper is at least 1.30 mg/day. In a still further embodiment, the dosage of copper is ranging from 1.250 to 10 mg/day, for example from 1.30 to 2 mg/day, for example from 1.30 to 1.50 mg/day.

In one embodiment, the dosage of copper is at least 1.46 mg/day. In a further embodiment, the dosage of copper is at least 1.48 mg/day. In a still further embodiment, the dosage of copper is ranging from 1.46 to 10 mg/day, for example from 1.46 to 2 mg/day, for example from 1.48 to 1.50 mg/day.

In one embodiment, the dosage of copper for a lactating woman is at least 1.46 mg/day. In a further embodiment, the dosage of copper is at least 1.48 mg/day. In a still further embodiment, the dosage of copper is ranging from 1.46 to 10 mg/day, for example from 1.46 to 2 mg/day, for example from 1.48 to 1.50 mg/day.

The copper may be comprised in any form suitable for ingestion by a woman such as a pregnant woman, a woman trying to conceive or a lactating woman. For example, copper may be comprised in the form of copper oxide, copper chloride, copper gluconate, copper sulfate, copper amino acid chelates or a mixture thereof.

In one embodiment, the dosage of zinc may be about 5-40, 7-13 or 9.5-12 mg/day.

In one embodiment, the dosage of zinc is at least 9.5 mg/day. In a further embodiment, the dosage of zinc is at least 10 mg/day. In a still further embodiment, the dosage of zinc is ranging from 9.5 to 12 mg/day, for example from 9.5 to 11.5 mg/day, for example from 10 to 11 mg/day.

In one embodiment, the dosage of zinc for a lactating woman is at least 9.5 mg/day. In a further embodiment, the dosage of zinc is at least 10 mg/day. In a still further embodiment, the dosage of zinc is ranging from 9.5 to 12 mg/day, for example from 9.5 to 11.5 mg/day, for example from 10 to 11 mg/day.

The zinc may be comprised in any form suitable for ingestion by a woman such as a pregnant woman, a woman trying to conceive or a lactating woman. For example, zinc may be comprised in the form of zinc acetate, zinc chloride, zinc citrate, zinc gluconate, zinc lactate, zinc oxide, zinc sulfate, zinc carbonate or a mixture thereof.

In one embodiment, the dosage of selenium may be about 20-400, 25-250, 26-85 or 60-70 μg/day.

In one embodiment, the dosage of selenium is at least 131 μg/day. In a further embodiment, the dosage of selenium is at least 135 μg/day. In a still further embodiment, the dosage of selenium is ranging from 131 to 400 μg/day, for example from 140 to 250 μg/day, for example from 150 to 200 μg/day.

In one embodiment, the dosage of selenium for a lactating woman is at least 131 μg/day. In a further embodiment, the dosage of selenium is at least 135 μg/day. In a still further embodiment, the dosage of selenium is ranging from 131 to 400 μg/day, for example from 140 to 250 μg/day, for example from 150 to 200 μg/day.

The selenium may be comprised in any form suitable for ingestion by a woman such as a pregnant woman, a woman trying to conceive or a lactating woman. For example, selenium may be comprised in the form of sodium selenite, sodium hydrogen selenite or a mixture thereof.

In one embodiment, the dosage of calcium is about 100-2500, 500-2000 or 1000-1500 mg/day.

In one embodiment, the dosage of calcium is at least 750 mg/day. In a further embodiment, the dosage of calcium is at least 850 mg/day. In a still further embodiment, the dosage of calcium is ranging from 750 to 2500 mg/day, for example from 850 to 2000 mg/day, for example from 900 to 1500 mg/day.

In one embodiment, the dosage of calcium for a lactating woman is at least 750 mg/day. In a further embodiment, the dosage of calcium is at least 850 mg/day. In a still further embodiment, the dosage of calcium is ranging from 750 to 2500 mg/day, for example from 850 to 2000 mg/day, for example from 900 to 1500 mg/day.

In one embodiment, the dosage of calcium is at least 860 mg/day. In a further embodiment, the dosage of calcium is at least 900 mg/day. In a still further embodiment, the dosage of calcium is ranging from 860 to 2500 mg/day, for example from 900 to 2000 mg/day, for example from 900 to 1500 mg/day.

In one embodiment, the dosage of calcium for a lactating woman is at least 860 mg/day. In a further embodiment, the dosage of calcium is at least 900 mg/day. In a still further embodiment, the dosage of calcium is ranging from 860 to 2500 mg/day, for example from 900 to 2000 mg/day, for example from 900 to 1500 mg/day.

The calcium may be comprised in any form suitable for ingestion by a woman such as a pregnant woman, a woman trying to conceive or a lactating woman. For example, calcium may be comprised in the form of calcium citrate, calcium carbonate or a mixture thereof.

In one embodiment, the dosage of phosphorous is about 70-4000, 100-1500 or 250-1250 mg/day.

In one embodiment, the dosage of phosphorus is at least 1275 mg/day. In a further embodiment, the dosage of phosphorus is at least 1300 mg/day. In a still further embodiment, the dosage of phosphorus is ranging from 1300 to 4000 mg/day, for example from 1300 to 2000 mg/day, for example from 1300 to 1500 mg/day.

In one embodiment, the dosage of phosphorus for a lactating woman is at least 1275 mg/day. In a further embodiment, the dosage of phosphorus is at least 1300 mg/day. In a still further embodiment, the dosage of phosphorus is ranging from 1300 to 4000 mg/day, for example from 1300 to 2000 mg/day, for example from 1300 to 1500 mg/day.

In one embodiment, the dosage of phosphorus is at least 1250 mg/day. In a further embodiment, the dosage of phosphorus is at least 1275 mg/day. In a still further embodiment, the dosage of phosphorus is ranging from 1250 to 4000 mg/day, for example from 1275 to 2000 mg/day, for example from 1300 to 1500 mg/day.

In one embodiment, the dosage of phosphorus for a lactating woman is at least 1250 mg/day. In a further embodiment, the dosage of phosphorus is at least 1275 mg/day. In a still further embodiment, the dosage of phosphorus is ranging from 1250 to 4000 mg/day, for example from 1275 to 2000 mg/day, for example from 1300 to 1500 mg/day.

The phosphorous may be comprised in any form suitable for ingestion by a woman such as a pregnant woman, a woman trying to conceive or a lactating woman. For example, phosphorous may be comprised in the form of sodium phosphate.

In a further embodiment of the present invention, the dosage of iron is ranging from 9.5 to 60 mg/day, for example from 9.5 to 30 mg/day, for example 9.5 to 20 mg/day; the dosage of managese is ranging from 2.1 to 4 mg/day, for example from 2.3 to 3.5 mg/day; the dosage of magnesium is ranging from 270 to 350 mg/day, for example from 300 to 350 mg/day; the dosage of copper is ranging from 1.250 to 10 mg/day, for example from 1.30 to 2 mg/day, for example from 1.30 to 1.50 mg/day; the dosage of calcium is ranging from 750 to 2500 mg/day, for example from 850 to 2000 mg/day, for example from 900 to 1500 mg/day; and the dosage of phosphorus is ranging from 1300 to 4000 mg/day, for example from 1300 to 2000 mg/day, for example from 1300 to 1500 mg/day. In such embodiment, the subject receiving the mineral combination or composition comprising it is for example a lactating woman.

Vitamins, Fatty Acids and Proteins

The minerals disclosed herein may be used in combination with further agents, in particular vitamin E, n-3 fatty acids (preferably selected from the group consisting of docosahexaenoic acid (DHA) and 18:3 n-3 octadecatrienoic acid (alpha-linolenic acid)) and/or a protein selected from the group consisting of alpha-lactalbumin, lactoferrin and albumin.

In another aspect, the invention provides an n-3 fatty acid for use in treating or preventing mastitis in a subject, preferably wherein the fatty acid is selected from the group consisting of docosahexaenoic acid (DHA) and 18:3 n-3 octadecatrienoic acid (alpha-linolenic acid).

In another aspect, the invention provides a protein selected from the group consisting of alpha-lactalbumin, lactoferrin and albumin for use in treating or preventing mastitis in a subject.

Such agents (vitamin E, n-3 fatty acids, alpha-lactalbumin, lactoferrin and albumin) may be used in any form suitable for ingestion by animals, preferably humans (e.g. are non-toxic). The agents may be used, for example in compositions such as nutritional compositions, in any appropriate amount. The skilled person will be able to determine appropriate amounts depending on the desired dosage of the agent. Dosages may depend on factors such as the age, size and health status of the woman to whom they are administered, on her lifestyle, as well as on her genetic heritage. Dosages may be in line with the recommended daily intakes (RDA) developed by organisations such as the Food and Nutrition Board of the National Academy of Sciences.

In one embodiment, the dosage of vitamin E is about 11-1000, 7.5-300 or 11-19 mg/day.

In one embodiment, the dosage of vitamin E is at least 8.1 mg/day. In a further embodiment, the dosage of phosphorus is at least 8.5 mg/day. In a still further embodiment, the dosage of phosphorus is ranging from 8.1 to 300 mg/day, for example from 8.5 to 19 mg/day, for example from 9.5 to 19 mg/day.

In one embodiment, the dosage of vitamin E for a lactating woman is at least 8.1 mg/day. In a further embodiment, the dosage of phosphorus is at least 8.5 mg/day. In a still further embodiment, the dosage of phosphorus is ranging from 8.1 to 300 mg/day, for example from 8.5 to 19 mg/day, for example from 9.5 to 19 mg/day.

The vitamin E may be, for example, in the form of a tocopherol or a mixture of different tocopherols. For example, the vitamin E may be alpha-tocopherol, gamma-tocopherol or a mixture of alpha-tocopherol and gamma-tocopherol.

The vitamin E may be comprised in any form suitable for ingestion by a woman such as a pregnant woman, a woman trying to conceive or a lactating woman, for example, alpha-tocopherol and/or gamma-tocopherol, and/or may be comprised in the form of tocopherol concentrate mix, L-vitamin E, D,L-vitamin E, tocopherols mixed pure, D,L-alpha-tocopherol, D,L-alpha tocopheryl acetate, tocopherol rich extract or a mixture thereof.

In one embodiment, the vitamin E is alpha-tocopherol.

In one embodiment, the dosage of docosahexaenoic acid (DHA) is less than or equal to 1000 mg/day, preferably about 500-1000 mg/day.

In one embodiment, the dosage of alpha-linolenic acid is less than or equal to 2000 mg/day, preferably about 500-1000 mg/day.

In one embodiment, the dosage of phosphatidylcholine is about 1500-1750 mg/day.

In one embodiment, the dosage of lecithin is about 1500-1750 mg/day.

In one embodiment, the dosage of lactoferrin is about 5-500 mg/day, preferably about 100-500 mg/day.

With respect to dosages defined herein as amounts per daily dose the amount of nutrient in a composition administered to the subject may vary depending upon whether it is intended to be consumed once a day, or more or less frequently.

Methods of Treatment

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

It is to be appreciated that all references herein to treatment include curative, palliative and prophylactic treatment. The treatment of mammals, particularly humans, is preferred. Both human and veterinary treatments are within the scope of the invention.

The minerals, fatty acids, proteins, combinations and compositions disclosed herein may be administered to a woman desiring to get pregnant, to a pregnant woman and/or to a lactating woman.

If administration is to a woman desiring to get pregnant, administration may be for example during at least 1, 2, 3 or 4 months preceding the pregnancy or desired pregnancy.

If administration is to a pregnant woman, administration may be for example for at least 4, at least 8, at least 12, at least 16, at least 20, at least 24, at least 28 or at least 36 weeks during pregnancy. As the nutritional requirements increase in the second and third trimester of pregnancy, it may be particularly beneficial if administration is throughout the second and/or third trimester of pregnancy.

Administration pre-pregnancy and/or during pregnancy may enable a woman to build up a store of one or more of the minerals, fatty acids and/or proteins before lactation.

If administration is to a lactating woman, administration may be for example for any part of the lactation period for example up to 2 years, up to 1 year, up to 9, 8, 7, 6, 5, 4, 3, 2 or 1 months post birth.

In one embodiment, administration is to a woman desiring to get pregnant, to a pregnant woman and/or to a lactating woman.

Composition

The term “maternal nutritional composition” as used herein refers to any composition that has been specifically manufactured for consumption by a pregnant woman, a woman trying to conceive or a lactating woman, or a composition that is specifically marketed at pregnant women, women trying to conceive or lactating (e.g. breast-feeding) women.

The maternal nutritional composition may be, for example, a food product, a functional food product, a drink (beverage), a dairy product or dairy substitute product, a pharmaceutical formulation or a supplement.

The term “dairy product” as used herein refers to food products produced from animals such as cows, goats, sheep, yaks, horses, camels and other mammals. Examples of dairy products are low-fat milk (e.g. 0.1%, 0.5% or 1.5% fat milk), fat-free milk, milk powder, whole milk, whole milk products, butter, buttermilk, buttermilk products, skim milk, lactose-free products, high milk-fat products, condensed milk, crème fraiche, cheese, ice cream and confectionery products, probiotic drinks or probiotic yoghurt-type drinks. A dairy substitute product may be a soya, almond or vegetable-based dairy substitute, e.g. a milk or yoghurt substitute.

The term “pharmaceutical formulation” as used herein refers to a composition comprising at least one pharmaceutically-active agent, chemical substance or drug. The pharmaceutical formulation may be in solid or liquid form and can comprise at least one additional active agent, carrier, vehicle, excipient or auxiliary agent identifiable by the skilled person. The pharmaceutical formulation may be in the form of a tablet, capsule, granules, powder, liquid or syrup.

The term “beverage product” as used herein refers to a nutritional product in liquid or semi-liquid form that may be safely consumed by an individual. The beverage product may be a water-based product, such as a product in which the agents of the invention are dissolved or suspended in water.

The term “food product” as used herein refers to any kind of product that may be safely consumed by a woman, in particular a pregnant woman, a woman trying to conceive or a lactating (e.g. breast-feeding) woman. Said food product may be in solid, semi-solid or liquid form and may comprise one or more nutrients, foods or nutritional supplements. For example, the food product may further comprise one or more of the following nutrients and micronutrients: a source of protein, a source of lipid, a source of carbohydrate, vitamins and minerals. The composition may also contain anti-oxidants, stabilisers (when provided in solid form) or emulsifiers (when provided in liquid form).

The term “functional food product” as used herein refers to a food product providing an additional health-promoting or disease-preventing function to the individual. Food products and functional food products include, for example, cereal-based products, yoghurts or other milk-derived products and bars.

The term “supplement” as used herein refers to a nutritional product that provides nutrients (e.g. vitamins and/or minerals) to an individual that may otherwise not be consumed in sufficient quantities by said individual. Supplements may be, for example, provided in the form of a pill, a tablet, a lozenge, a chewy capsule or tablet, a capsule, or a powder supplement that can be, for example, dissolved in water or milk, or sprinkled on food. Supplements typically provide selected nutrients without providing a significant portion of the overall nutritional needs of a subject. Typically supplements do not represent more than 0.1%, 1%, 5%, 10% or 20% of the daily energy need of a subject. In the context of the present invention the subject may be, for example, a woman trying to get pregnant, a pregnant woman and/or a lactating woman.

The term “pregnancy supplement” as used herein refers to a supplement that is specifically formulated for administration to a woman who is trying to conceive and/or to a woman who is pregnant, or marketed towards a woman who is trying to conceive and/or a woman who is pregnant.

The term “lactation supplement” as used herein refers to a supplement that is specifically formulated for administration to a woman who is lactating, or marketed toward a woman who is lactating. Consumption of lactation supplements may be advised to commence during pregnancy.

The compositions of the invention may also comprise ingredients commonly used in maternal nutritional compositions. Non-limiting examples of such ingredients include: probiotics, lipids, carbohydrates, pharmaceutically-active agents and conventional additives, such as anti-oxidants, stabilisers, emulsifiers, acidulants, thickeners, buffers or agents for pH adjustment, chelating agents, colourants, excipients, flavour agents, osmotic agents, pharmaceutically-acceptable carriers, preservatives, sugars, sweeteners, texturisers, emulsifiers and water.

It may also be beneficial if the compositions of the invention comprise probiotics. Probiotics may help nutrients pass through the gut.

The term “probiotic” as used herein refers to live probiotic bacteria, non-replicating probiotic bacteria, dead probiotic bacteria, non-viable probiotic bacteria, fragments of probiotic bacteria, such as DNA, metabolites of probiotic bacteria, cytoplasmic compounds of probiotic bacteria, cell wall materials of probiotic bacteria, culture supernatants of probiotic bacteria, and combinations of any of the foregoing.

The probiotic may be live probiotic bacteria, non-replicating probiotic bacteria, dead probiotic bacteria, non-viable probiotic bacteria and any combination thereof.

Subject

The term “subject” as used herein refers to either a human or non-human animal. The non-human animal may be, for example, a livestock animal or a companion animal.

A “companion animal” is any domesticated animal, and includes, without limitation, cats, dogs, rabbits, guinea pigs, ferrets, hamsters, mice, gerbils, horses, cows, goats, sheep, donkeys, pigs and the like.

In one embodiment, the subject is a human subject. In another embodiment, the subject is a companion animal. Preferably, the subject is a human.

In one embodiment, the subject is at risk of mastitis and/or subclinical mastitis. In another embodiment, the subject is a lactating animal.

In one embodiment, the human subject is a woman.

In a further embodiment, the human subject is a lactating woman. In another embodiment, the human subject is a pregnant woman.

In a still further embodiment, the human subject is a woman at risk of mastitis and/or of subclinical mastitis.

In another embodiment, the human subject is a lactating woman at risk of mastitis and/or of subclinical mastitis.

Treating and Preventing

The term “prevent” as used herein includes prevention and reducing the risk of a condition.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature.

### EXAMPLES

### Example 1

Methods

Within the framework of a multicentre European observational study to characterise the human milk (HM) composition in the first four months of lactation (Atlas of Human Milk Nutrients study), we set out to understand whether there are differences in the HM composition between lactating women with subclinical mastitis (SCM) versus those without based on the sodium/potassium (Na/K) ratios in the HM.

Study Protocol

The ATLAS study was conducted in seven countries across Europe (France, Italy, Norway, Portugal, Romania, Spain and Sweden) as a longitudinal, observational, cohort in which HM as well as multiple maternal and infant parameters were collected at six time points post-partum (0-3 d, 17±3 d, 30±3 d, 60±5 d, 90±5 d and 120±5 d). Institutional and local Ethical boards of each centre approved the study. The participants provided a written informed consent form to participate in the study after receiving explanations and having read and understood the purpose and the objective of the study in their respective local languages. Pregnant women were recruited before delivery, generally during the last trimester of pregnancy. Inclusion criteria for this study were: (a) pregnant women between ages of 18 and 40 years; (b) BMI between 19 and 29, inclusive; (c) intention to breastfeed at least until 4 months post-partum; and (d) agreement to the study protocol and signed informed consent form. Exclusion criteria for this study were: (a) currently participating in another trial; (b) presenting conditions that contraindicate breastfeeding; (c) medical conditions or on medications for conditions such as metabolic and cardiovascular abnormalities; (d) dietary probes such as anorexia or bulimia; and (e) subjects not able to comply to the study procedures. Dedicated, trained and certified research nurses and assistants collected all data for this study. Maternal data included: demography, anthropometry, medical history, history of dietary supplements and three-day food diaries. Infant data included: demography, anthropometry, history of medication use, body composition (one centre in France and one in Sweden) and infant intake diary (three centres in France only).

Standardised Human Milk Sampling

HM sampling was standardised for all subjects. Milk was collected at 11 h00±2 h00 using an electric breast pump (Medela Symphony). For each mother, milk was collected from the same breast for the entire study and mothers were requested to empty the breast in the previous feed. This collected single full breast milk samples were mixed and an aliquot of 10-40 mL HM for each time point was collected. For colostrum, or the first time point 5-10 mL was collected. The remainder of the HM was returned to the mother for feeding to the infant at a later time point, if so required. Each collected HM sample was transferred to freezing tubes, labelled with subject number and collection information, stored at −18° C. in the home freezer, transferred to the hospital for storage at −80° C. and then shipped on dry ice to the Nestle Research Centre (Lausanne, Switzerland) where it was stored at −80° C. until analysis. The frozen HM samples were thawed once for aliquoting into 15 individual small volume fractions (0.2 mL to 2 mL) in separate polypropylene tubes dedicated to the different analyses.

Assessment of SCM Status

Lactating women were categorised in to two groups: those having any SCM (defined as Na/K ratio>0.6) and those normal (defined as Na/K ratio≤0.6) based on the Na/K ratios in HM in early lactation (days 2, 17 and 30). Lactating women having at least 1 instance of SCM during any of these three time points were classified as having any SCM, while those in the normal category did not have any instance of SCM in any of these time points.

Fatty Acid Quantification in HM

Fatty acid profiles were determined by preparing the methyl esters of fatty acids (FAMEs). A direct transesterification of HM was performed with methanolic chloridric acid solution as described by Cruz-Hernandez et al. (Cruz-Hernandez, C. et al. (2017) J Sep Sci 40: 3289-3300). Briefly, into a 10 mL screw cap glass test tube, milk (250 μL) was added and mixed with 300 μL of internal standard FAME 11:0 solution (3 mg/mL) and 300 μL of internal standard TAG 13:0 solution (3 mg/mL). After addition of 2 mL of methanol, 2 mL of methanolic chloridric acid (3 N) and 1 mL of hexane, the tubes were heated at 100° C. for 90 min. To stop the reaction 2 mL of water was added and after centrifugation (1200 g×5 min) the upper phase (hexane) was transferred into gas chromatography vials. The analysis of FAMEs was performed by GC using a CP-Sil 88 capillary column (100 m, 0.25 mm id. 0.25 μm film thickness) and their identification by comparison of retention time with authentic standards (GC standard Nestlè 36 from NuCheck-Prep, Elysan Minn. USA).

Protein Quantification in HM

Total protein content in HM was measured using the colorimetric bicinchoninic acid (BCA) method according to the protocol provided with the BCA assay kit (ThermoFisher Scientific). The four major HM proteins alpha-lactalbumin, lactoferrin, serum albumin and caseins were quantified using a LabChip system as described previously (Affolter et al. (2016) Nutrients 8: 504).

Mineral Quantification in HM

Quantification of minerals was realised using Inductively Coupled Plasma Mass Spectrometry (ICP-MS).

For Sodium (Na), Magnesium (Mg), Phosphorous (P), Potassium (K), Calcium (Ca), Manganese (Mn), Iron (Fe), Copper (Cu), Zinc (Zn) and Selenium (Se), 0.7 mL of human breast milk was transferred into PFA vessels and mineralised in a OEM® Microwave digestion system using HNO₃/H₂O₂. Mineralised samples were transferred to PE tubes, diluted with MQ water and Germanium (Ge) and Tellurium (Te) were added as internal standards. Quantification was realised by ICP-MS using He as collision gas.

Certified Reference Materials (CRM) were added to all analytical series to control the quality of the quantification.

Results

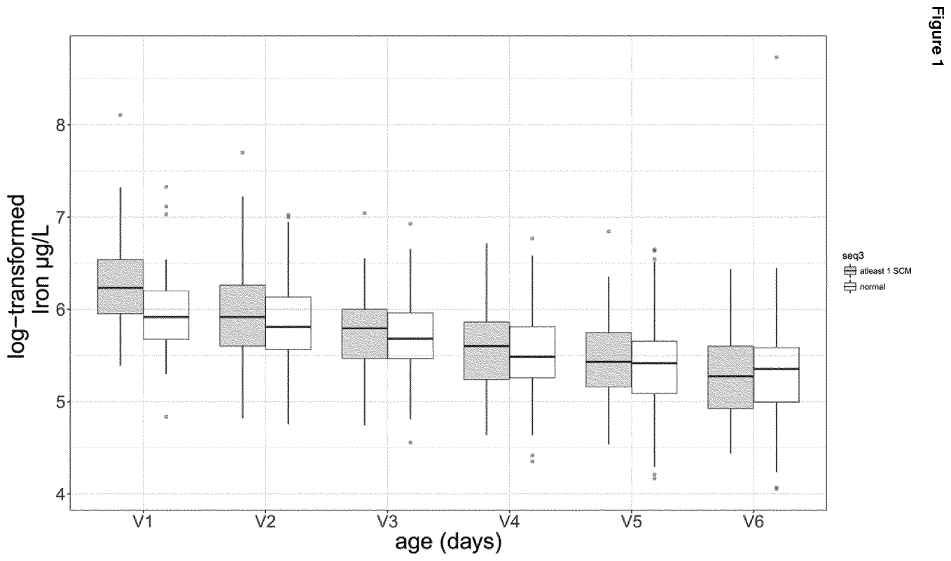

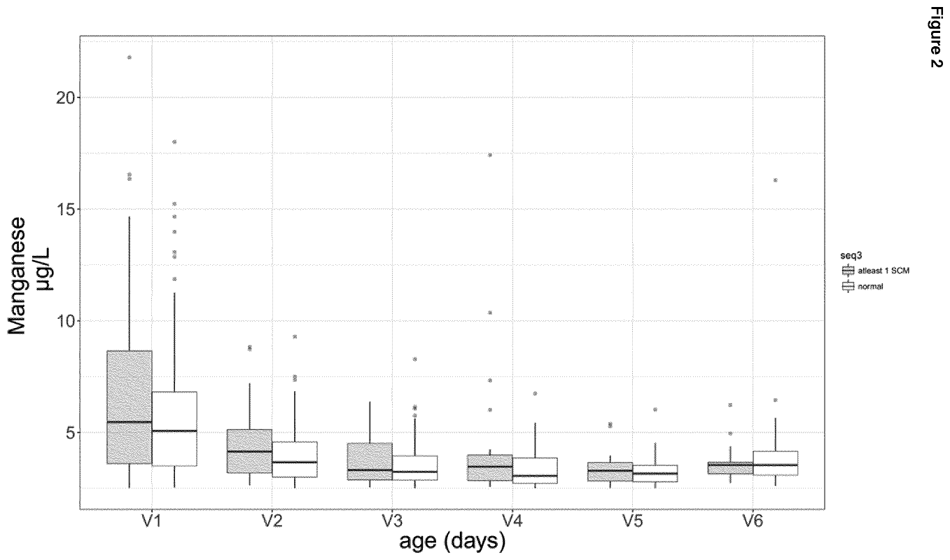

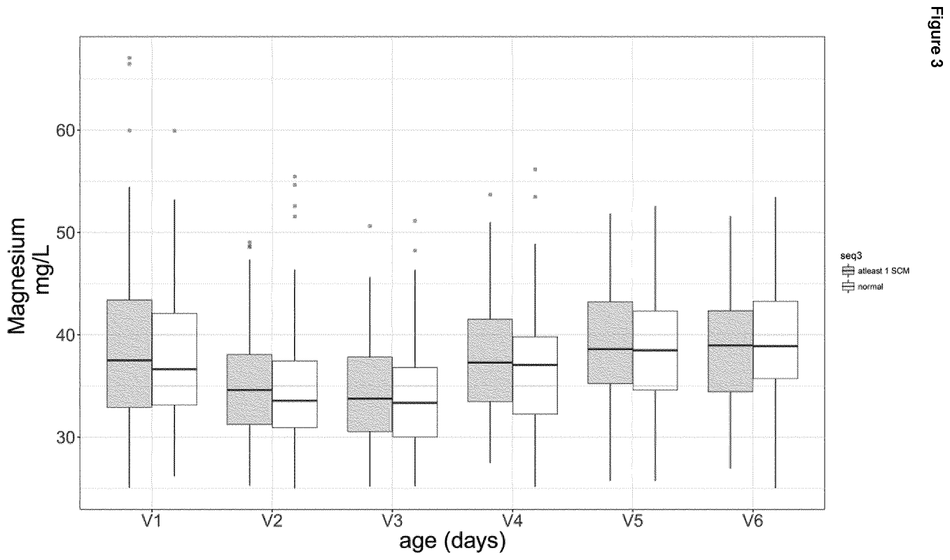

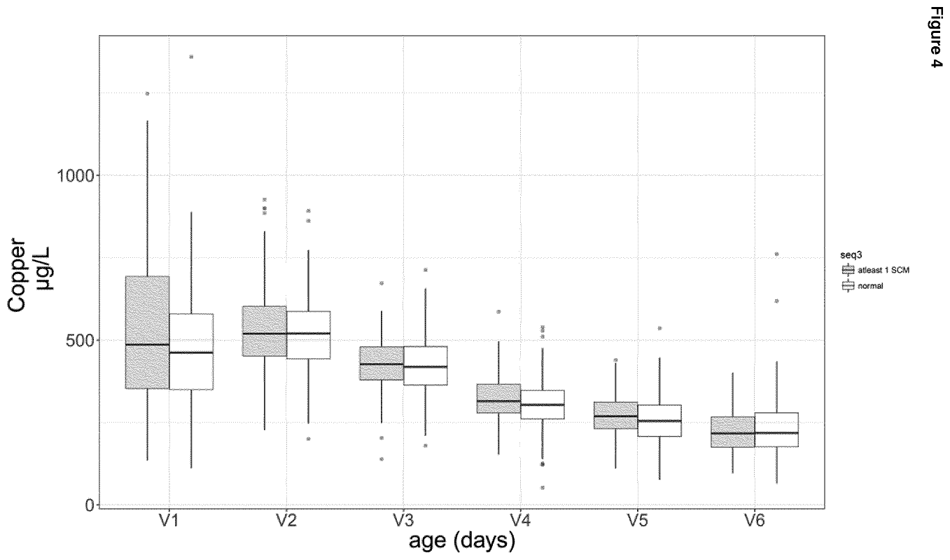

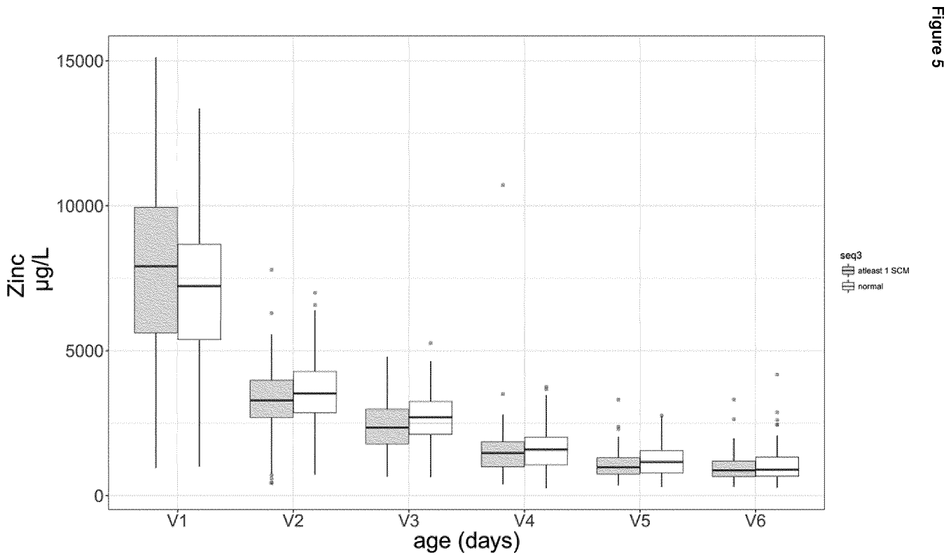

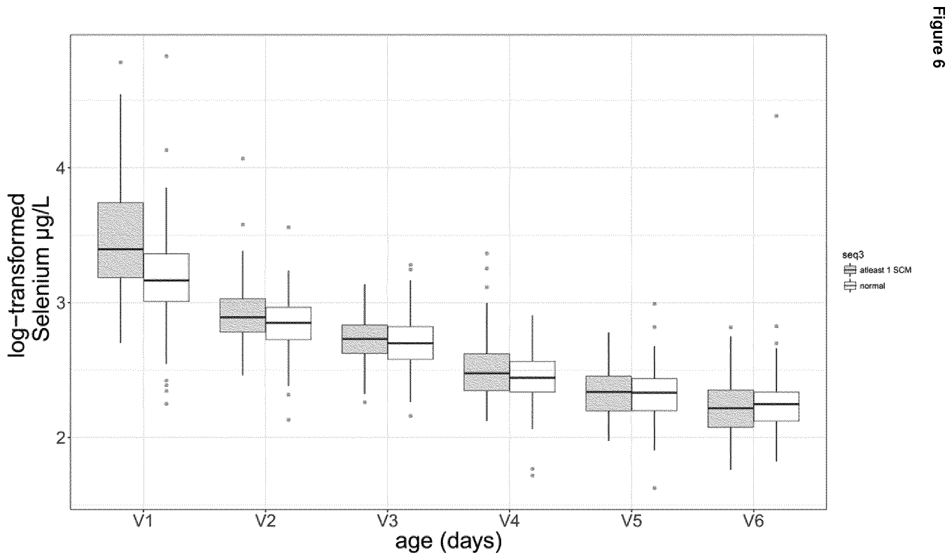

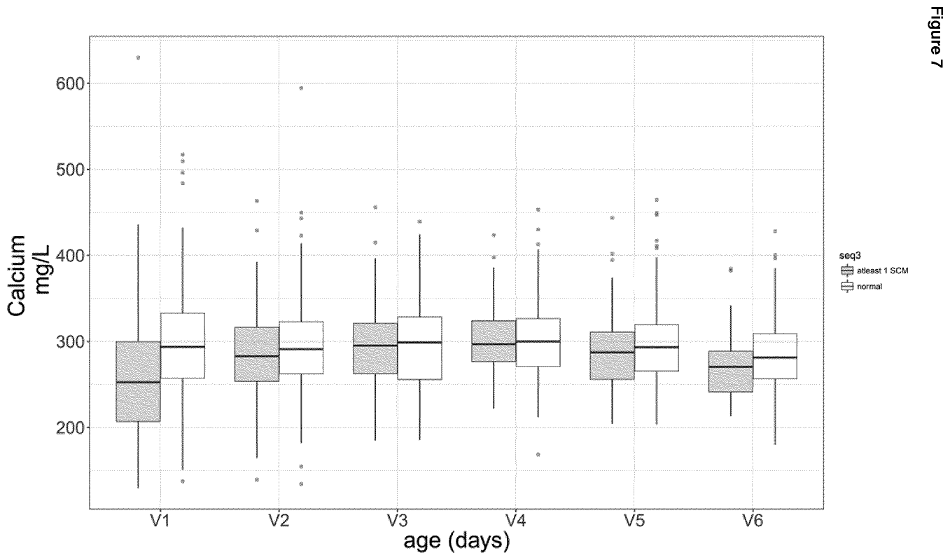

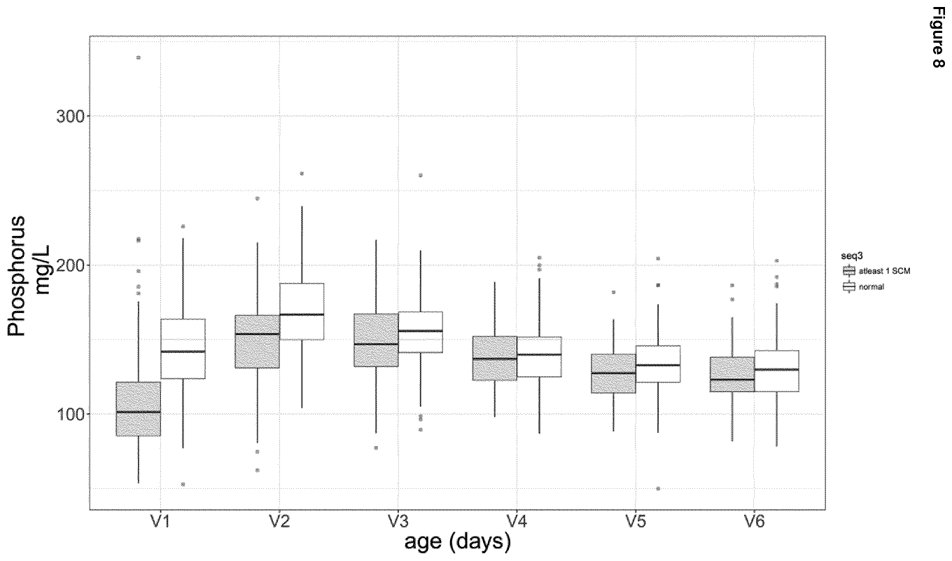

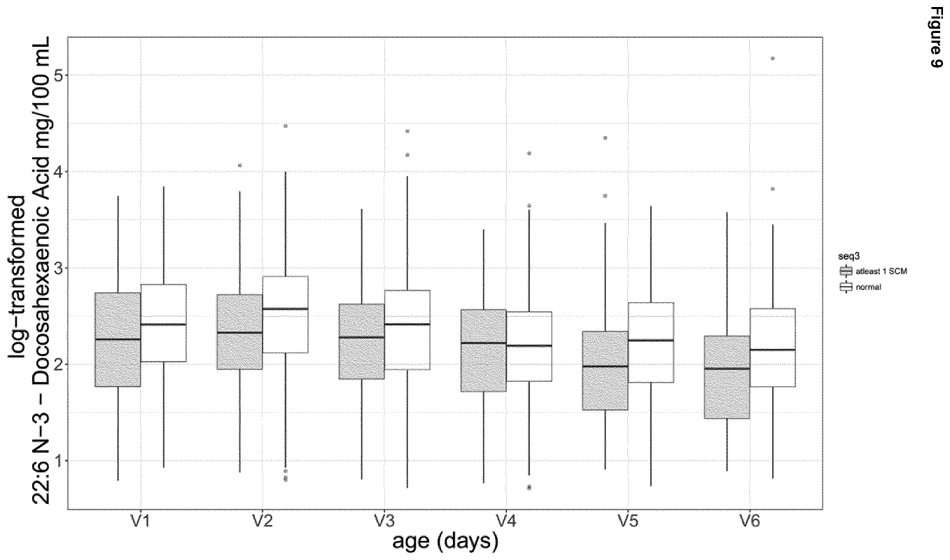

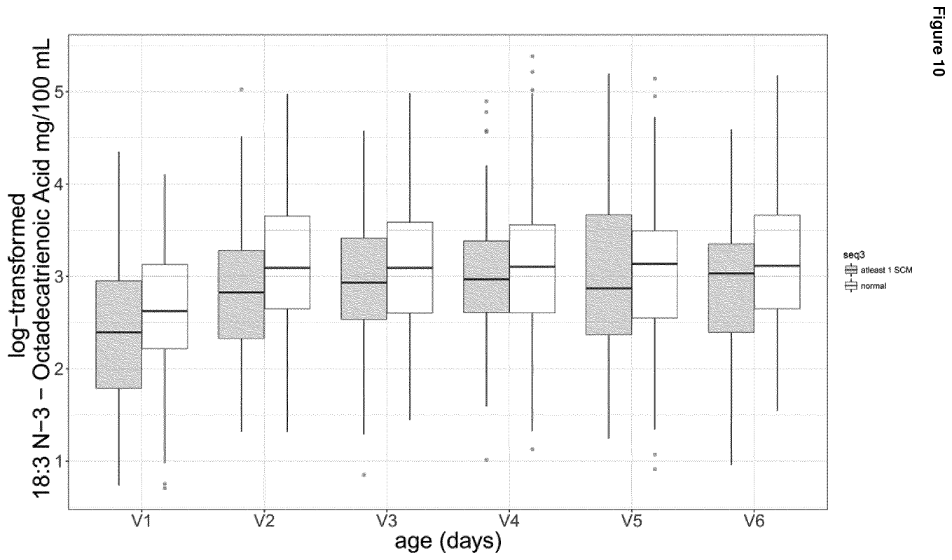

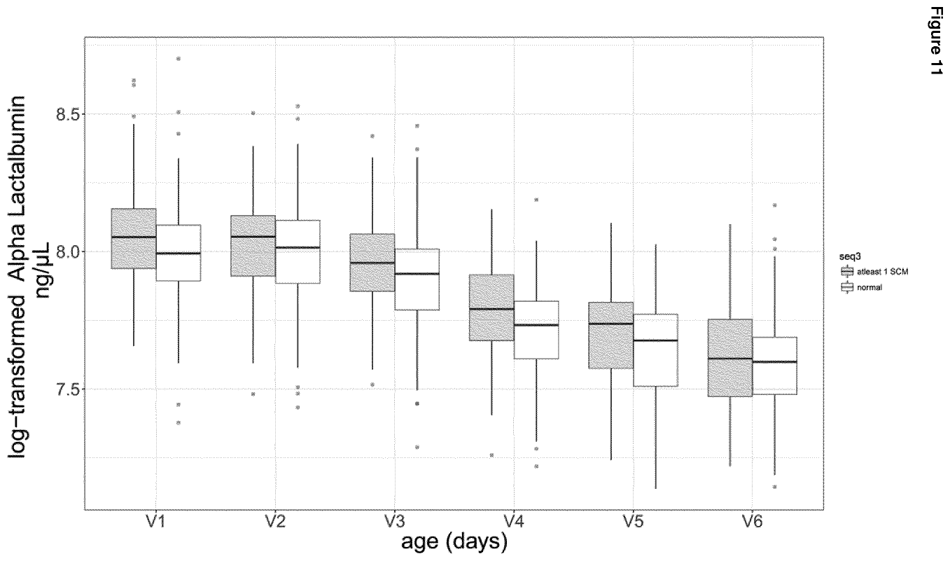

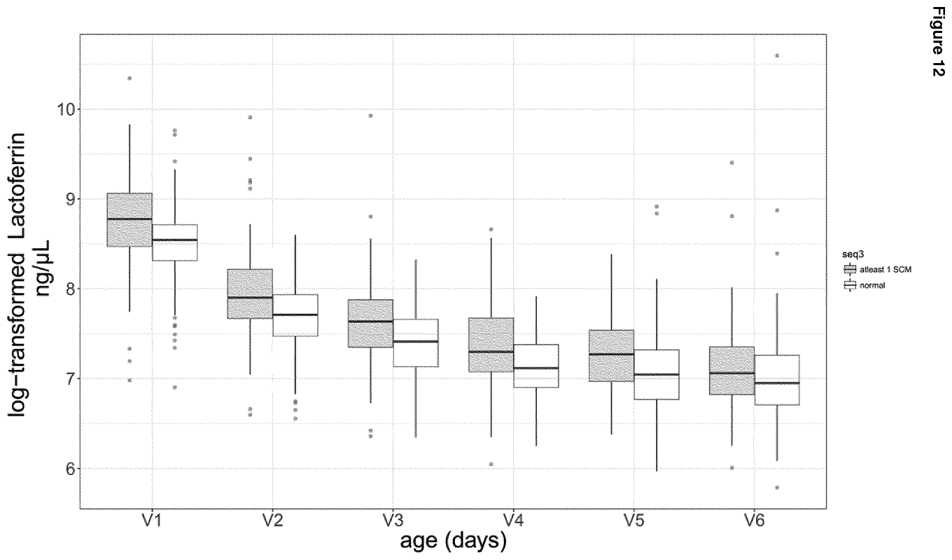

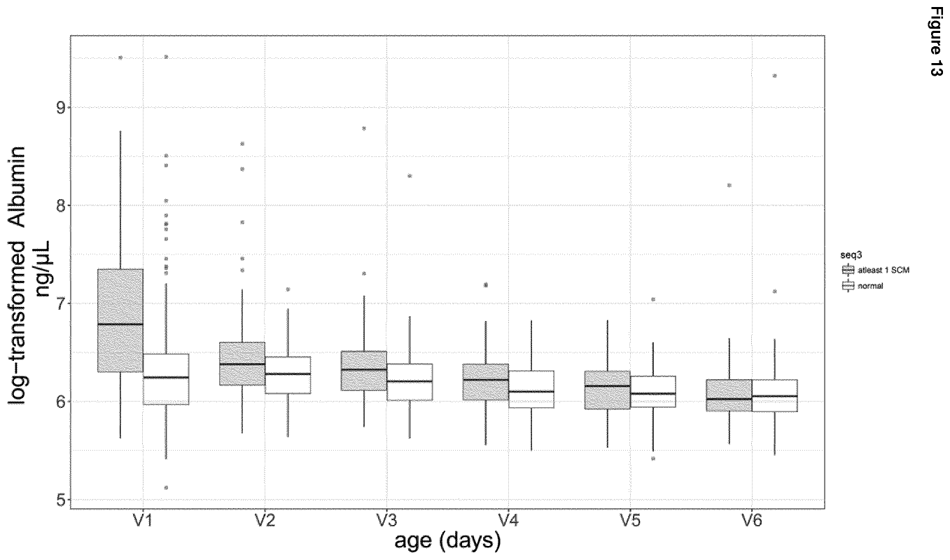

The concentrations of iron, manganese, magnesium, copper, zinc, selenium, calcium, phosphorous, DHA, 18:3 n-3 octadecatrienoic acid, alpha-lactalbumin, lactoferrin and albumin in both the milk of mothers with sub-clinical mastitis and the milk of normal mothers at the 6 time-points post-partum are shown in FIGS. 1-13 and Tables 1-3.

Women with sub-clinical mastitis have higher concentrations of iron, manganese, magnesium, copper, zinc and selenium; and lower concentrations of calcium and phosphorous in their milk in comparison to normal women.

The n-3 fatty acids docosahexaenoic acid (DHA) and 18:3 n-3 octadecatrienoic acid (alpha-linolenic acid) are present at lower concentrations in the milk of women with sub-clinical mastitis in comparison to normal women.

In addition, alpha-lactalbumin, lactoferrin and albumin are present at higher concentrations in the milk of women with sub-clinical mastitis in comparison to normal women.

TABLE 1
Mineral and trace element concentrations of human milk across lactation
by presence or absence of sub-clinical mastitis (SCM).

| | | | | | | | | Overall |
|---|---|---|---|---|---|---|---|---|
| | | Day 2 | Day 17 | Day 30 | Day 60 | Day 90 | Day 120 | P value |
| Iron | Any SCM | 601 ± 392 | 461 ± 310 | 346 ± 162 | 292 ± 141 | 264 ± 140 | 216 ± 103 | <0.0001 |
| (μg/L) | | (n = 91) | (n = 97) | (n = 84) | (n = 78) | (n = 74) | (n = 71) | |
| | Normal | 415 ± 193 | 389 ± 186 | 326 ± 137 | 278 ± 132 | 245 ± 127 | 262 ± 502 | |
| | | (n = 111) | (n = 192) | (n = 174) | (n = 166) | (n = 156) | (n = 148) | |
| Manganese | Any SCM | 6.6 ± 3.9 | 4.4 ± 1.6 | 3.7 ± 1.1 | 4.5 ± 3.4 | 3.4 ± 0.8 | 3.7 ± 0.9 | <0.0001 |
| (μg/L) | | (n = 79) | (n = 41) | (n = 26) | (n = 22) | (n = 19) | (n = 13) | |
| | Normal | 5.8 ± 3.1 | 3.9 ± 1.3 | 3.5 ± 1.0 | 3.4 ± 0.9 | 3.3 ± 0.7 | 4.0 ± 2.2 | |
| | | (n = 92) | (n = 103) | (n = 75) | (n = 45) | (n = 36) | (n = 37) | |
| Magnesium | Any SCM | 38.5 ± 8.5 | 35.0 ± 6.2 | 34.2 ± 5.5 | 37.4 ± 5.8 | 38.8 ± 5.4 | 38.4 ± 5.4 | 0.076 |
| (mg/L) | | (n = 87) | (n = 94) | (n = 80) | (n = 76) | (n = 72) | (n = 69) | |
| | Normal | 38.0 ± 6.5 | 34.3 ± 5.7 | 33.9 ± 5.2 | 36.5 ± 5.6 | 38.6 ± 5.9 | 39.3 ± 6.2 | |
| | | (n = 106) | (n = 171) | (n = 149) | (n = 162) | (n = 154) | (n = 145) | |
| Copper | Any SCM | 533 ± 267 | 529 ± 135 | 423 ± 86 | 325 ± 74 | 268 ± 72 | 221 ± 69 | <0.001 |
| (μg/L) | | (n = 91) | (n = 97) | (n = 84) | (n = 78) | (n = 74) | (n = 71) | |
| | Normal | 472 ± 175 | 522 ± 111 | 422 ± 89 | 304 ± 76 | 256 ± 77 | 228 ± 89 | |
| | | (n = 111) | (n = 192) | (n = 174) | (n = 166) | (n = 156) | (n = 148) | |
| Zinc | Any SCM | 7975 ± 3093 | 3234 ± 1274 | 2422 ± 906 | 1616 ± 1208 | 1113 ± 521 | 996 ± 520 | <0.0001 |
| (μg/L) | | (n = 91) | (n = 97) | (n = 84) | (n = 78) | (n = 74) | (n = 71) | |
| | Normal | 7178 ± 2734 | 3590 ± 1051 | 2686 ± 872 | 1599 ± 692 | 1219 ± 571 | 1031 ± 563 | |
| | | (n = 111) | (n = 192) | (n = 174) | (n = 166) | (n = 153) | (n = 148) | |
| Selenium | Any SCM | 37 ± 19 | 19 ± 6 | 15 ± 3 | 12 ± 3 | 10 ± 2 | 9 ± 2 | <0.0001 |
| (μg/L) | | (n = 91) | (n = 97) | (n = 84) | (n = 78) | (n = 74) | (n = 71) | |
| | Normal | 26 ± 12 | 18 ± 3 | 15 ± 3 | 12 ± 2 | 10 ± 2 | 10 ± 6 | |
| | | (n = 111) | (n = 192) | (n = 174) | (n = 166) | (n = 156) | (n = 148) | |
| Calcium | Any SCM | 256 ± 73 | 281 ± 54 | 296 ± 47 | 299 ± 41 | 288 ± 45 | 271 ± 36 | 0.003 |
| (mg/L) | | (n = 91) | (n = 97) | (n = 84) | (n = 78) | (n = 74) | (n = 71) | |
| | Normal | 295 ± 72 | 293 ± 55 | 296 ± 51 | 301 ± 45 | 297 ± 48 | 284 ± 42 | |
| | | (n = 111) | (n = 192) | (n = 174) | (n = 166) | (n = 156) | (n = 148) | |
| Phosphorus | Any SCM | 109 ± 41 | 150 ± 31 | 149 ± 29 | 138 ± 21 | 128 ± 18 | 126 ± 20 | <0.0001 |
| (mg/L) | | (n = 91) | (n = 97) | (n = 84) | (n = 78) | (n = 74) | (n = 71) | |
| | Normal | 144 ± 32 | 169 ± 28 | 155 ± 24 | 139 ± 21 | 133 ± 20 | 131 ± 22 | |
| | | (n = 111) | (n = 192) | (n = 174) | (n = 166) | (n = 156) | (n = 148) | |

TABLE 2
Fatty acid concentrations of human milk across lactation by presence or absence of sub-clinical mastitis (SCM).

| | | | | | | | | Overall |
|---|---|---|---|---|---|---|---|---|
| | | Day 2 | Day 17 | Day 30 | Day 60 | Day 90 | Day 120 | P value |
| DHA | Any SCM | 11.5 ± 7.8 | 12.6 ± 9.0 | 10.5 ± 5.8 | 10.3 ± 6.1 | 9.5 ± 10.0 | 8.4 ± 5.9 | 0.0001 |
| (mg/100 mL) | | (n = 103) | (n = 103) | (n = 89) | (n = 78) | (n = 77) | (n = 74) | |
| | Normal | 13.7 ± 8.8 | 15.1 ± 10.2 | 13.6 ± 11.0 | 11.3 ± 8.4 | 10.8 ± 6.9 | 11.7 ± 15.8 | |
| | | (n = 158) | (n = 187) | (n = 168) | (n = 155) | (n = 144) | (n = 137) | |
| 18:3 n-3 | Any SCM | 13.9 ± 11.2 | 21.8 ± 19.4 | 24.5 ± 18.7 | 27.8 ± 24.7 | 27.3 ± 27.6 | 23.6 ± 18.8 | <0.0001 |
| octadecatrienoic | | (n = 100) | (n = 104) | (n = 90) | (n = 80) | (n = 77) | (n = 76) | |
| acid | Normal | 17.0 ± 11.3 | 29.1 ± 21.9 | 29.3 ± 23.7 | 32.8 ± 33.8 | 27.9 ± 24.7 | 32.3 ± 30.4 | |
| (mg/100 mL) | | (n = 159) | (n = 187) | (n = 171) | (n = 161) | (n = 154) | (n = 144) | |

TABLE 3
Protein concentrations of human milk across lactation by presence or absence of sub-clinical mastitis (SCM).

| | | | | | | | | Overall |
|---|---|---|---|---|---|---|---|---|
| | | Day 2 | Day 17 | Day 30 | Day 60 | Day 90 | Day 120 | P value |
| Alpha- | Any SCM | 3232.8 ± 685.0 | 3131.2 ± 548.4 | 2893.1 ± 506.6 | 2469.0 ± 466.5 | 2249.5 ± 422.3 | 2089.4 ± 415.8 | <0.0001 |
| lactalbumin | | (n = 102) | (n = 104) | (n = 91) | (n = 81) | (n = 77) | (n = 75) | |
| (ng/μL) | Normal | 3043.6 ± 597.1 | 3036.6 ± 544.4 | 2740.7 ± 499.1 | 2278.1 ± 372.4 | 2126.7 ± 374.1 | 2015.8 ± 378.0 | |
| | | (n = 128) | (n = 187) | (n = 170) | (n = 162) | (n = 156) | (n = 147) | |
| Lactoferrin | Any SCM | 7053.9 ± 3832.6 | 3315.6 ± 2486.1 | 2430.8 ± 2176.9 | 1757.1 ± 955.4 | 1543.1 ± 699.2 | 1510.4 ± 1502.3 | <0.0001 |
| (ng/μL) | | (n = 102) | (n = 104) | (n = 91) | (n = 81) | (n = 77) | (n = 75) | |
| | Normal | 5340.2 ± 2372.7 | 2379.4 ± 913.4 | 1765.9 ± 680.7 | 1339.0 ± 485.6 | 1298.6 ± 810.1 | 1468.2 ± 3269.1 | |
| | | (n = 128) | (n = 187) | (n = 170) | (n = 162) | (n = 156) | (n = 147) | |
| Albumin | Any SCM | 1534.6 ± 1821.9 | 729.2 ± 671.1 | 659.1 ± 655.3 | 536.5 ± 190.5 | 474.7 ± 130.2 | 483.8 ± 388.8 | <0.0001 |
| (ng/μL) | | (n = 102) | (n = 104) | (n = 91) | (n = 81) | (n = 77) | (n = 75) | |
| | Normal | 813.3 ± 1342.5 | 551.8 ± 160.6 | 529.7 ± 301.8 | 469.2 ± 123.3 | 455.8 ± 126.8 | 516.8 ± 894.1 | |
| | | (n = 128) | (n = 187) | (n = 170) | (n = 162) | (n = 156) | (n = 147) | |

### Example 2

Methods

Study Population

This study used data from ‘ATLAS’, a longitudinal, observational study across seven European countries between December 2012 and January 2016. The study was approved by the institutional and local ethical boards for each center and was registered at ClincalTrials.gov with identifier NCT01894893. Maternal and infant demographics, anthropometry, and medical history were collected by trained and certified research nurses and assistants.

Human milk was collected from 305 women in 7 European countries. Of those, 185 provided information on dietary intake. 8 women were further excluded due to missing information, resulting in a final sample size of 177 women. Written informed consent was obtained from all women in their respective local languages.

SCM Analysis

Milk samples were obtained using an electric breast pump (Medela Symphony, Switzerland) from the same breast throughout the study period, at 11:00 h±2:00 h to avoid circadian influence. Samples were first frozen at −18° C. until delivery to the Nestle Research Centre (Lausanne, Switzerland) and then at −80° C. for further analysis.

SCM was assessed in early lactation, at visits 1 (0-3 days postpartum), 2 (17 days postpartum ±3 days), and 3 (30 days postpartum ±3 days). SCM was defined as having a sodium potassium ratio (Na/K) in breastmilk higher than 0.6 at any of the three visits. Moderate SCM was defined as Na/K ratio between >0.6 and ≤1 while severe SCM as Na/K ratio>1.

Dietary Intake Data

Dietary intake was assessed with 3-day food diaries at visits 2 (V2) and 3 (V3). The dietary information was then translated to nutrient and food group intakes by Nutrilog using the French food group classification and nutrient composition database (CIQUAL).

Diets which contained less than 1074.8 kcal or more than 4776.9 kcal of energy were considered outliers and were removed. After removing the outlier diets from the dataset, we considered each visit in V2-V3 in turn and the subset of subjects who attended that visit. For each visit, we removed a subject and all associated dietary information from a given visit if fewer than two non-outlier diets were reported for that subject across the three-day survey period for that visit. For example, if a subject S1 attended visit V2 and reported one outlier diet and one non-outlier diet for that visit, then we removed S1 and all her reported diets for V2 from the dataset. 177 subjects who attended at least one visit were retained for analysis.

Once the mean daily consumption was calculated, it was then normalized by the mean daily energy intake for that visit in kcal/day. This adjusted consumption was then averaged over all visits that each individual subject attended within the subset. As a final step, we then performed normalization to zero mean and standard deviation of 1. An example pipeline for a subject who attended all visits in S2 (visits V2-V3)

Dietary reference values for nutrients intake for lactating women were extracted from European Food Safety Authority (EFSA)'s summary report on Dietary Reference Values for nutrients.

Statistical Analysis

1) We used multivariable regression to examine the association between nutrient intake in relation to SCM. Wilcox-test was used to calculate the p-values.

Analyses were run with R.

Results are reported in Table 4.

2) We also used multivariable regression to examine the association between nutrient intake in relation to SCM. The statistical model has SCM status, Country and Mode of Delivery as covariates and contrast estimates were calculated to show the differences between SCM group and the No SCM group. A logarithmic transformation was applied as the nutrient intake data generally has a skewed distribution.

Analyses were run with the statistical software R ver 3.2.1 and packages mass and contrast were used for modelling and estimation.

Results are reported in Table 5.

Results

Table 4 reports the median intake of certain nutrients for women with SCM (i.e. those with human milk sodium potassium (Na/K) ratio>0.6 during any of the following visits: days 2, 17, and 30), and for women with no SCM (i.e. those with no SCM are defined as having a Na/K ratio≤0.6 during any of the following visits: days 2, 17, and 30).

These data show that in the group of women with subclinical mastitis certain minerals (namely iron, manganese, magnesium, copper, calcium, phosphorous) were present at lower amounts in the diet as compared to the diet of women not having subclinical mastitis.

Similarly, data show that in the group of women with subclinical mastitis certain vitamin E was present at lower amounts in the diet as compared to the diet of women not having subclinical mastitis.

Table 5 reports the median intake of zinc and selenium for women with SCM (i.e. those with human milk sodium potassium (Na/K) ratio>0.6 during any of the following visits: days 2, 17, and 30), and for women with no SCM (i.e. those with no SCM are defined as having a Na/K ratio≤0.6 during any of the following visits: days 2, 17, and 30).

These data show that in the group of women with subclinical mastitis certain minerals in addition to those previously mentioned (namely zinc and selenium) were present at lower amounts in the diet as compared to the diet of women not having subclinical mastitis.

These findings thus provide additional evidence that supplementation of one or more of such nutrients in the diet can prevent and/or treat subclinical mastitis.

TABLE 4

| | Median intakes of energy | | | |
|---|---|---|---|---|
| | and nutrients | | | |
| | Women with | Women with | | |
| | SCM* at | SCM at | | Difference in |
| | any visit | no visit | wilcox- | median intakes |
| Average diet | n = 52 | n = 125 | test | between SCM |
| of V2-V3 | Median | Median | p value | and no SCM |
| Magnesium, mg | 265 | 313 | 0.01 | −48.003 |
| Phosphorus, mg | 1254 | 1340 | 0.01 | −85.926 |
| Calcium, mg | 744 | 974 | 0.00 | −229.719 |
| Manganese, mg | 2 | 3 | 0.02 | −0.498 |
| Iron, total, mg | 9 | 11 | 0.06 | −1.632 |
| Copper, mg | 1.2 | 1.4 | 0.01 | −0.213 |
| Vitamin E, mg | 8 | 10 | 0.02 | −1.467 |

TABLE 5

| | Median intakes of energy | | | |
|---|---|---|---|---|
| | and nutrients | | | |
| | Women with | Women with | | |
| | SCM* at | SCM at | | |
| | any visit | no visit | | Difference |
| | n=52 | n = 125 | | in geometric |
| | Estimated | Estimated | | mean intakes |
| Average diet | Geometric | Geometric | Contrast | between SCM |
| of V2-V3 | Mean | Mean | p value | and no SCM |
| Zinc, mg | 9.46 | 10.84 | 0.0325 | −1.39 |
| Selenium, μg | 130.96 | 156.64 | 0.0064 | −25.68 |

All publications mentioned in the above specification are herein incorporated by reference. Various modifications and variations of the disclosed agents, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for treating or preventing mastitis in a subject comprising the step of administering a composition comprising a mineral selected from the group consisting of iron, manganese, magnesium, and a combination of two of more thereof to a subject in need of same.

2. The method according to claim 1, wherein the combination comprises iron and manganese.

3. The method according to claim 1, wherein the mineral is in combination with one or more further minerals selected from the group consisting of copper, zinc, selenium, calcium and phosphorus.

4. The method according to claim 1, wherein the mineral is in combination with vitamin E.

5. The method according to claim 1, wherein the combination comprises iron, manganese, copper, zinc, selenium and vitamin E.

6. The method according to claim 1, wherein the mineral is in combination with:
a) an n-3 fatty acid;
b) a protein selected from the group consisting of alpha-lactalbumin, lactoferrin and albumin; and/or
c) phosphatidylcholine and/or lecithin.

7. Method for treating or preventing mastitis in a subject comprising the step of administering a composition comprising an n-3 fatty acid to a subject in need of same.

8. Method for treating or preventing mastitis in a subject comprising the step of administering a composition comprising a protein selected from the group consisting of alpha-lactalbumin, lactoferrin and albumin to a subject in need of same.

9. Method according to claim 1, wherein the composition is a maternal nutritional composition, for use during lactation and/or pregnancy.

10. Method according to claim 1, wherein the mastitis is sub-clinical mastitis or clinical mastitis.

11. Method according to claim 1, wherein the subject is at risk of suffering from sub-clinical mastitis or clinical mastitis.

12. Method according to claim 1, wherein the treatment or prevention increases the probability of initiating and/or continuing breastfeeding by the subject.

13. Method according to claim 1, wherein the subject is able to breast-feed for at least 4 months.

14. Method according to claim 1, wherein the treatment or prevention increases the quality and/or quantity of the subject's breast milk.

15-16. (canceled)
